(19) United States
(12) Patent Application Publication
(10) Pub. No.: US 2009/0247973 A1
(43) Pub. Date: Oct. 1, 2009

(54) ANTIBACTERIAL NANO-SILVER CONTAINED POLYMER ABSORBENT AND SANTITARY ARTICLES THEREOF

(75) Inventors: An-Chi Yeh, Kaohsiung County (TW); Deng-Nan Horng, Kaohsiung County (TW); Chia-Feng Liang, Kaohsiung County (TW); Kuo-Hui Wu, Taoyuan County (TW); Fu-Chu Yang, Tainan City (TW)

Correspondence Address: APEX JURIS, PLLC, 12733 LAKE CITY WAY NORTHEAST, SEATTLE, WA, 98125, US

(73) Assignee: LIN HORN TECHNOLOGY CO., LTD., CHIA YI HSIEN (TW)

(21) Appl. No.: 12/056,866

(22) Filed: Mar. 27, 2008

(51) Int. Cl. A61F 13/53; A61K 33/38; A41D 13/11

(52) U.S. Cl. 604/360; 424/618; 128/863; 977/778

(57) ABSTRACT

An antibacterial nano-silver contained polymer absorbent and sanitary articles thereof includes a polymer absorbent and an antibacterial material coated thereon that has a plurality of supporting elements adsorbed nano-silver. The antibacterial nano-silver contained polymer absorbent is adopted on sanitary articles to be in contact with secretion generating portions of users to directly absorb secretions of user's body or prevent microbe-contained external air from entering the user's body to provide disinfecting effect and a healthy condition for the user.

### BACKGROUND OF THE INVENTION

1. Field of the Invention

The present invention relates to an antibacterial polymer absorbent and sanitary articles thereof and particularly to a nano-silver contained polymer absorbent and sanitary articles thereof.

2. Description of the Prior Art

Silver ions (Ag⁺) have powerful bacteria suppressing and disinfecting effect, and also have strong bacteria thwarting activity, thus are widely used in healthcare products to provide sterilization function. However, silver-contained antibacterial compounds are effective only when silver ions are released in a pathogenic environment.

Metal silver can be oxidized to become active silver ions through interaction with water molecules. In the event that the silver is held in a porous material release of the silver ions lasts longer. This phenomenon can expand applications of the silver-contained material.

Nowadays, many antibacterial medicines are made from organic materials which are not suitable to be used in compounds that have to last a longer durability. However, silver-contained inorganic material can overcome such a constraint. At present zeolite, calcium phosphorate and fibers have been developed to contain silver to become inorganic antibacterial materials, especially silver-contained silica, such as quartz glass and silica films. They have excellent durability and antibacterial activity, thus are hotly pursued for disinfecting application.

But to date there are still not many personal sanitary articles made from the silver-contained antibacterial material previously discussed. Sterilizing gauze masks made from the silver-contained antibacterial material also are not available on the market. There are still rooms for improvement.

### SUMMARY OF THE INVENTION

In view of the aforesaid problems the present invention aims to provide a silver-contained polymer absorbent for sanitary articles that has a supporting element with a selected content of silver dispensed on the surface thereof.

The silver-contained polymer absorbent according to the invention has nano-silver to provide disinfecting effect. It includes a polymer absorbent and an antibacterial material adsorbing to the polymer absorbent material. The antibacterial material consists of a plurality of supporting elements adsorbing the nano-silver.

In one aspect the antibacterial material is formed in powder at a diameter smaller than 10 μm.

In another aspect the polymer absorbent is made from non-woven fabrics or cotton fabrics.

In yet another aspect the supporting elements are active carbon, bamboo carbon, Kaolin clay or Montmo-rillonite.

In yet another aspect the sanitary articles according to the invention include a body which provides a structure to wear or hold a sanitary article and a nano-silver contained polymer absorbent fastened to the body to be in contact with a user's secretion outlet. The nano-silver contained polymer absorbent includes a polymer absorbent and an antibacterial material.

In yet another aspect the sanitary articles may be female sanitary pads, diapers, sanitary napkins or gauze masks.

The nano-silver contained polymer absorbent according to the invention provides a longer durability and stronger disinfecting effect, thus is especially desirable for use on personal sanitary articles to keep users in a bacteria free condition for a longer period of time.

The foregoing, as well as additional objects, features and advantages of the invention will be more readily apparent from the following detailed description, which proceeds with reference to the accompanying drawings.

### BRIEF DESCRIPTION OF THE DRAWINGS

The above and other objects of the present invention will become readily apparent by reference to the following detailed description when considered in conjunction with the accompanying drawings wherein:

FIG. 1A is a schematic view of an embodiment of a supporting element of the antibacterial material of the invention;

FIG. 1B is a schematic view of an embodiment of nano-silver of the antibacterial material of the invention;

FIG. 1C is a schematic view of an embodiment of the nano-silver adsorbed to the supporting element of the antibacterial material; and

FIGS. 2A and 2B are schematic views of an embodiment of the antibacterial material of the invention showing the processes of binding the polymer material and the antibacterial material.

### DESCRIPTION OF THE PREFERRED EMBODIMENTS

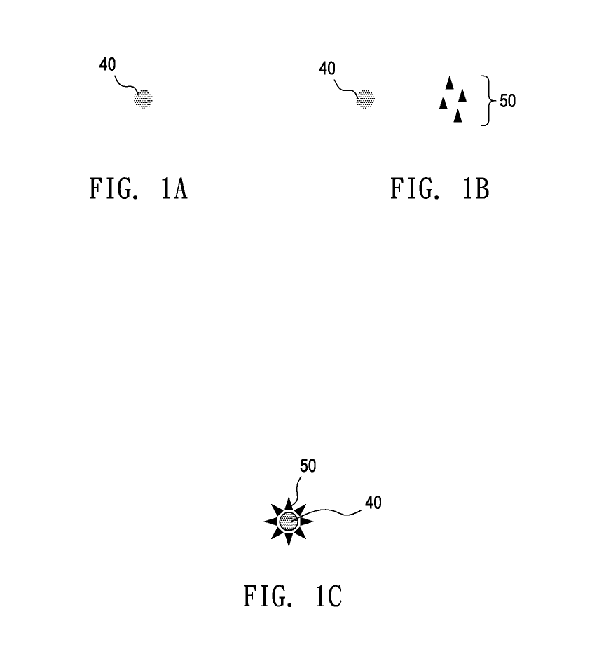

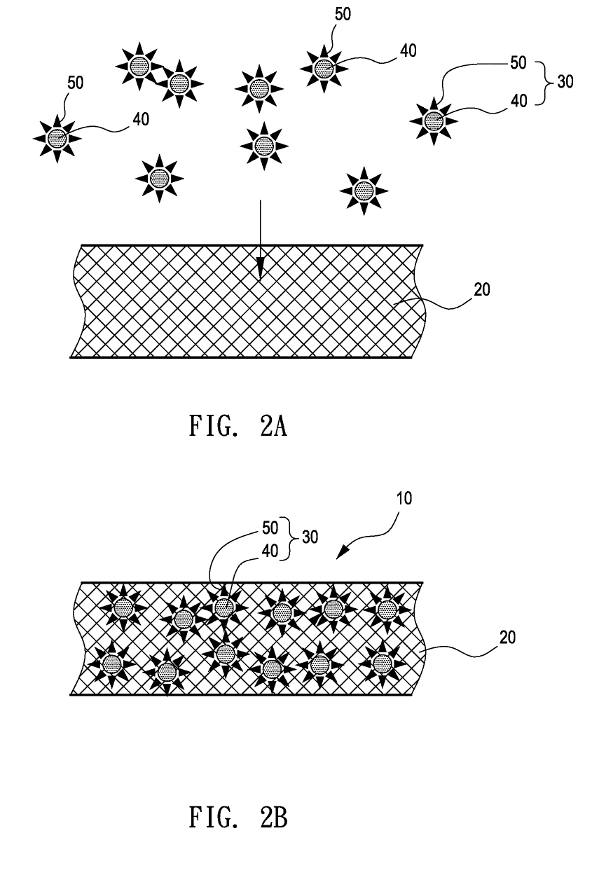

Pleases refer to FIGS. 1A through 2B for embodiments of components and fabrication processes of the antibacterial nano-silver contained polymer absorbent of the invention. The nano-silver contained polymer absorbent 10 includes a polymer absorbent 20 and an antibacterial material 30. The polymer absorbent 20 may be made from non-woven fabrics or cotton fabrics. The antibacterial material 30 has a supporting element 40 which may be made from active carbon, bamboo carbon, Kaolin clay or Montmo-rillonite. The antibacterial material 30 is adsorbed to the polymer absorbent 20, and includes a plurality of supporting elements 40 adsorbing nano-silver 50.

In the processes depicted in FIGS. 2A and 2B, the supporting element is first activated by a surface active agent; next, the activated supporting element is submerged in a bi-ammonia silver nitrate solution at room temperature and mixed for a selected duration and diluted; reaction takes place for a number of hours at the room temperature under ammonia gas; finally, clean and separate through de-ionized water and ethanol, and dry in vacuum to become the antibacterial material in a powder form.

The nano-silver contained polymer absorbent thus made can be directly used on sanitary articles at locations in contact with user's body, especially secretion outlets such as respiratory or excretory organs to absorb a great amount of liquid and secretions. The antibacterial material can prohibit breeding of bacteria (for instance on sanitary articles such as sanitary pads, diapers, sanitary napkins or the like). The polymer material is air permeable and allows air to pass through. The antibacterial material can filter out microbes in the air so that users can inhale cleaner air (such as used on gauze masks).

As a conclusion, the present invention provides a novel design and offers a significant improvement over the conventional products and techniques.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An antibacterial nano-silver contained polymer absorbent, comprising:
a polymer absorbent; and
an antibacterial material which is adsorbed to the polymer absorbent and includes a plurality of supporting elements that adsorb nano-silver.

2. The antibacterial nano-silver contained polymer absorbent of claim 1, wherein the antibacterial material is formed in powder at a diameter smaller than 10 μm.

3. The antibacterial nano-silver contained polymer absorbent of claim 1, wherein the polymer absorbent is selectively made from non-woven fabrics or cotton fabrics.

4. The antibacterial nano-silver contained polymer absorbent of claim 1, wherein the supporting elements are selected from the group consisting of active carbon, bamboo carbon, Kaolin clay and Montmo-rillonite.

5. A sanitary article to provide sanitary protection for a user, comprising:
a body to provide a structure to wear or hold the sanitary article; and
a nano-silver contained polymer absorbent which is fastened to the body and in contact with secretion outlets of the user, and includes a polymer absorbent and an antibacterial material.

6. The sanitary article of claim 5, wherein the body is a female sanitary pad.

7. The sanitary article of claim 5, wherein the body is a diaper or sanitary napkin.

8. The sanitary article of claim 5, wherein the body is a gauze mask.
